(19) United States
(12) Patent Application Publication
(10) Pub. No.: US 2019/0159714 A1
(43) Pub. Date: May 30, 2019

(54) GRIP STRENGTH DETECTION MECHANISM, EXERCISE APPARATUS PROVIDED WITH GRIP STRENGTH DETECTION MECHANISM, AND METHOD FOR USING EXERCISE APPARATUS

(71) Applicants: CATALOGHOUSE CO., LTD., Tokyo (JP); OHASHI CHISO LABORATORY CO., LTD., Tokyo (JP)

(72) Inventors: Tadanori NAGASU, Tokyo (JP); Kenji NAGATA, Tokyo (JP); Hideo HATA, Tokyo (JP); Hidekazu KATO, Tokyo (JP)

(21) Appl. No.: 16/066,856

(22) PCT Filed: Dec. 28, 2015
(86) PCT No.: PCT/JP2015/086504

§ 371 Date: Oct. 19, 2018

(51) Int. Cl. A61B 5/22; A63B 24/00; A61B 5/00; G01L 19/08

(52) U.S. Cl. CPC A61B 5/225; A63B 24/0062; A61B 5/7425; A63B 2024/0068; A61B 2562/0247; A61B 2560/0214; G01L 19/083

(57) ABSTRACT

Provided are a grip strength detection mechanism, an exercise apparatus provided with the same, and a method for using the same. The exercise apparatus is provided with: a grip part 12 comprising a cylindrical flexible member; sealing members 13, 16 which seal both ends of the grip part 12 to form a deformable sealed space 18 which is surrounded by the grip part 12; a pressure sensor 28 which is held by the sealing member 16 and detects the pressure within the sealed space 18; and a control unit which detects, as the pressure within the grip part, a reference grip strength which is not more than the maximum grip strength detected by the pressure sensor 28 in order to display, on a display unit 7, the grip strength of a user gripping the grip part 12.

### TECHNICAL FIELD

The present invention relates to a grip strength detection mechanism adaptable to an exercise apparatus, an exercise apparatus including the grip strength detection mechanism, and a method for using the exercise apparatus.

### BACKGROUND ART

Conventionally, in order to measure a grip strength, a method for gripping a pair of handles held to face each other and measuring a change in load between the handles by the principle of a spring has been used. Regarding a grip strength meter, Patent Documents 1 and 2 disclose a configuration in which a gripping part having a sealed space is formed by a flexible member and a change in pressure of the sealed space when the gripping part is gripped is measured to measure a grip strength.

Incidentally, it has been reported that a blood pressure can be lowered and a high blood pressure can be prevented by training with a grip strength management. Accordingly, it is also considered that the grip strength meter can be used not only for simply measuring the grip strength, but also an exercise apparatus used for such training or the like and operated based on the grip strength. However, a problem arises in that the conventional grip strength meter cannot be adapted to such training or the like.

Further, Patent Document 3 discloses a pronunciation type fingertip function recovery exercise apparatus in which a hinge portion is provided at a center portion of one circumferential edge in the short radial direction in the plan view of each of a pair of outer shell bodies in which a metal plate or synthetic resin is molded into a bowl shape having a concave portion to have a substantially oval shape in the plan view and an arcuate shape in the cross-sectional view, the outer shell bodies overlap each other while the concave portions face each other, the hinge portions are connected to be rotatable by a hinge pin (thus, the main body has a structure similar to bivalves and instrumental castanets), one end of a support column is fixed to the center of the concave portion of one outer shell body and is erected, a substantially center portion of a convex surface of an elastic plate having a suitable length formed in an arcuate shape is fixed to the other end of the support column, and one end portion of the elastic plate is fixed to the inside of the circumferential edge of the other outer shell body (the concave portion) while the outer shell body opens to the opposite circumferential edge of the hinge portion by a suitable dimension.

When an operation of squeezing or loosening the pronunciation type fingertip function recovery exercise apparatus is repeated while holding the apparatus in a palm, it is possible to promote the recovery of the grip strength function or the numbness of the hand due to the high blood pressure by activating the whole blood circulation as well as the palm or fingertips. Further, since the elastic plate is pressed and deformed to generate a light sound whenever the apparatus is gripped with the palm in use, it is possible to consciously recognize the use feeling and to use the apparatus for a long time without getting tired.

However, in the pronunciation type fingertip function recovery exercise apparatus disclosed in Patent Document 3, there were problems that the hardness of the elastic plate was difficult to be adjusted, exercise to grip could not be performed when the elastic plate was too hard to be deformed, and appropriate exercise could not be performed when the elastic plate was too soft. Further, there was also troublesome to replace the fixed elastic plate for each adjustment.

Further, there was a problem that an exercise using the pronunciation type fingertip function recovery exercise apparatus was needed for a long time in order to solve a high blood pressure.

Patent Document 1: Japanese Unexamined Patent Application, Publication No. 2000-193537

Patent Document 2: Japanese Unexamined Utility Model Application, Publication No. S54-25362

Patent Document 3: Japanese Unexamined Utility Model Application, Publication No. H06-52824

### DISCLOSURE OF THE INVENTION

### Problems to be Solved by the Invention

An object of the invention is to provide a grip strength detection mechanism adaptable to an exercise apparatus for various trainings intended to lower a blood pressure by exercise, an exercise apparatus including the grip strength detection mechanism, and a method for using the exercise apparatus.

The inventors have contrived a grip strength detection mechanism which forms a sealed space by sealing both ends of a cylindrical flexible member, detects a change in pressure of the sealed space using a pressure sensor while gripping the flexible member, detects the changed pressure as a predetermined grip strength, records and displays the grip strength, and is able to output an instruction for each exercise, an exercise apparatus including the grip strength detection mechanism, and a method for using the exercise apparatus.

### Means for Solving the Problems

Hereinafter, Means for Solving the problems are described together with Reference Numerals used in Mode for Carrying Out the Invention. It should be understood that Reference Numerals are intended to clarify the correspondence between the description of Claims and the description of Mode for Carrying Out the Invention and it goes without saying that Reference Numerals are not restrictively used for the interpretation of the technical scope of Claims of the invention.

A grip strength detection mechanism 1 of the invention includes:

a grip part 12 which is formed by a cylindrical flexible member;
sealing members 13 and 16 that seal both ends of the grip part 12 and form a deformable sealed space surrounded by the grip part 12;
a pressure sensor 28 which is held by the sealing member 13 and detects a pressure of the sealed space 18; and
a control unit 31 which displays a grip strength of a user who grips the grip part 12 on a display unit 7 by a pressure detected by the pressure sensor 28 (claim 1).

A battery casing having flanges 11A and 11A formed at both ends is provided inside the grip part 12, and both ends of the grip part are respectively pressed against the flanges 11A and 11A by the sealing members 13 and 16 so that the grip part 12 is sealed by the sealing member (claim 2).

Further, the control unit 31 sets a predetermined pressure equal to or smaller than a pressure detected by the pressure sensor as a reference grip strength based on the detected pressure by a maximum grip strength of a user who grips the grip part 12, and

displays a detection result in which the grip strength of the user who grips the grip part reaches the reference grip strength on the display unit (claim 3).

The reference grip strength may be set within a range of a certain height difference degree with respect to a pressure corresponding to the reference grip strength and the reference grip strength may be displayed on the display unit (claim 4).

Furthermore, the invention includes an exercise apparatus 10 with the grip strength detection mechanism 1 of the invention (claim 5).

Further, as a method for using the exercise apparatus 10 with the grip strength detection mechanism 1 (claims 1 to 4), it is possible to perform exercise of lowering a blood pressure by repeating an operation of gripping the grip part 12 with the reference grip strength for a certain time and releasing the gripping for a certain time (claim 6).

Further, as a method for using the exercise apparatus 10 with the grip strength detection mechanism 1 (claims 3 and 4) in which the control unit 31

sets and records the reference grip strength by a pressure equal to or smaller than a pressure detected by the pressure sensor 28 based on the detected pressure, and
displays the pressure detected in the sealed space as the reference grip strength,
in which the reference grip strength is set to about 30% of the maximum grip strength of the user, and
in which an operation of gripping the grip part 12 with a strength of about 30% of the maximum grip strength of the user for 2 minutes and releasing the gripping for 1 minute is performed twice by each of left and right hands and is performed four times in total, thereby lowering a blood pressure (claim 7).

### Effects of the Invention

The grip strength detection mechanism 1 of the invention is suitable for the exercise apparatus used for various training and the like.

Further, according to the grip strength detection mechanism 1 of the invention, since a movable portion can be remarkably decreased in size as compared with a case in which the grip strength is detected and measured by the principle of a spring, it is possible to realize a decrease in size and weight by a simple configuration. Further, when the pressure sensor 28 or the like is held by the sealing member 16, the grip strength detection mechanism can be decreased in size and simplified while having a substantially bar-shaped cylindrical shape on the whole and thus the usability can be remarkably improved.

Further, the grip strength detection mechanism can be used in various places in a portable manner and can be decreased in size and weight to be portable.

Further, when the operation of gripping the grip part 12 of the exercise apparatus 10 including the grip strength detection mechanism 1 of the invention for a certain time and releasing the gripping for a certain time is repeated, the muscles are stained when gripping the grip part 12 and are relaxed when releasing the gripping, so that the blood vessel expands at a stretch. When the training is repeated, the elasticity of the blood vessel is remarkably improved. As a result, a blood pressure is lowered and a high blood pressure is solved.

Further, according to the exercise apparatus 10 with the grip strength detection mechanism 1 of the invention, when the grip strength is maintained at about the reference grip strength which is about 30% of the maximum grip strength for a certain time, it is possible to safely relax the gripping muscles. Accordingly, it is possible to prevent and solve a high blood pressure by lowering a blood pressure.

Particularly, when the reference grip strength is set to about 30% of the maximum grip strength of the user, the blood circulation inside the blood vessel is activated, the cell inside the blood vessel is stimulated, and NO (nitric oxide) having a function of stabilizing a blood pressure is more efficiently secreted from the inner wall of the blood vessel by the stimulus.

### BRIEF DESCRIPTION OF THE DRAWINGS

FIG. 1 is a diagram illustrating an exercise apparatus 10 including a grip strength detection mechanism 1 according to a first embodiment of the invention, where FIG. 1(B) is a plan view and FIGS. 1(A) and 1(C) are side views.

FIG. 2 is a perspective view illustrating a use state of the exercise apparatus 10 including the grip strength detection mechanism 1 of FIG. 1.

FIG. 3 is a cross-sectional view and a partially enlarged cross-sectional view. FIG. 3(A) is a cross-sectional view of the exercise apparatus 10 including the grip strength detection mechanism 1 of FIG. 1. FIG. 3(B) is a partially enlarged cross-sectional view illustrating the arrangement of a pressure sensor 28.

FIG. 4 is a block diagram of the grip strength detection mechanism 1 of FIG. 1.

FIG. 5 is a flowchart illustrating a process relating to an operation of the exercise apparatus including the grip strength detection mechanism 1 of FIG. 1 and a process of a control unit of the grip strength detection mechanism 1.

FIG. 6 is a plan view of a display unit 7.

FIG. 7 is a plan view illustrating a method for using the exercise apparatus of the invention and a change of the display unit due to elapse of exercise time, where FIGS. 7(A) and 7(B) respectively illustrate different blinking directions of the display unit.

FIG. 8 is an exploded perspective view of the exercise apparatus 10 including the grip strength detection mechanism 1 of FIG. 1.

### PREFERRED MODE FOR CARRYING OUT THE INVENTION

### Embodiment

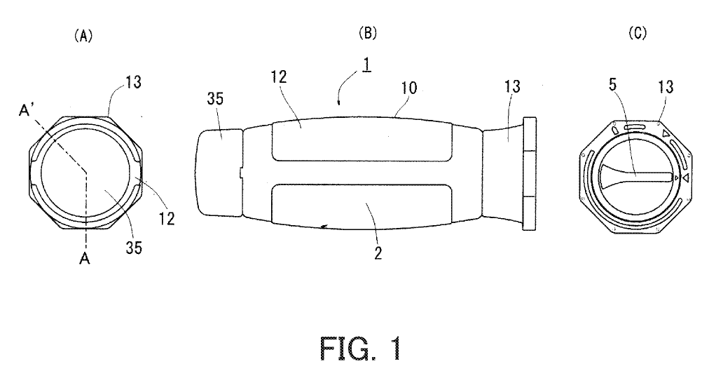

FIG. 1 illustrates an exercise apparatus 10 including a grip strength detection mechanism 1 according to an embodiment of the invention. The exercise apparatus 10 including the grip strength detection mechanism 1 is formed in a substantially cylindrical shape on the whole, a gripping part 2 is formed at a center portion of a center axial direction of the cylindrical shape, and cylindrical distal end portions 3 and 4 are respectively provided at both ends of the gripping part 2. Here, the gripping part 2 is formed in a barrel shape of which a diameter of a center portion is the largest, but instead of this, the gripping part may be formed to have the same diameter in the center axial direction, may be formed so that the diameter is the smallest at the center portion, and may be formed so that a cross-sectional shape of an outer peripheral surface in the direction along the center axis is formed as a wave shape to be familiar to hands.

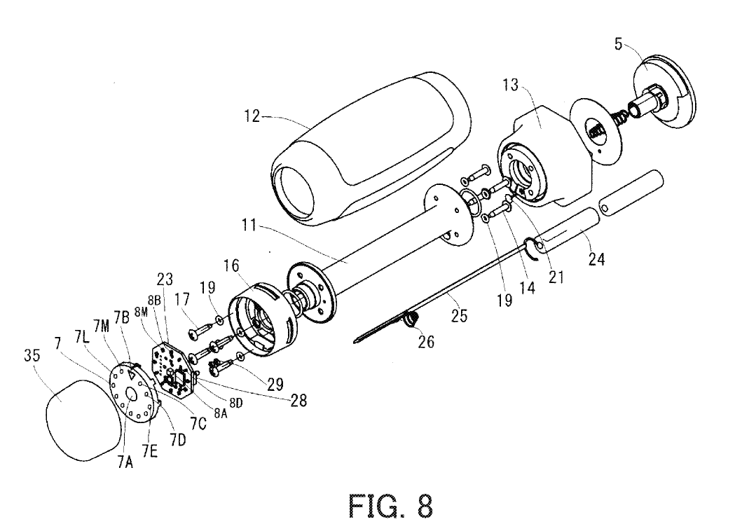

In the grip strength detection mechanism 1, the switch part 5 is disposed at the substantially center portion of the end surface of one distal end portion 4 and an operator of a power switch is formed by the switch part 5 so that the power is turned on when the switch part 5 is rotated in the clockwise direction. Further, a battery can be replaced after the switch part 5 is separated when the switch part 5 is rotated in the counter-clockwise direction. Further, in the grip strength detection mechanism 1, a display unit 7 is provided at the end surface of the other distal end portion 3. The display unit 7 is a bottomed cylindrical transparent or translucent member having a low circumferential wall height and is fitted into a top cap 16 to cover a wiring board 23 to be described later. Here, in the display unit 7, as illustrated in FIG. 8, a circular display unit 7A corresponding to a light emitting diode 8A provided at the center portion of the wiring board 23 is formed at the center portion and circular and triangular display units 7B to 7M corresponding to light emitting diodes 8B to 8M are concentrically formed at the intervals of an equal angle (about 30°) about the center display unit 7A. Here, in the display units 7B to 7M formed at the same angle intervals, only one display unit 7B of the display units 7B to 7M is formed in a triangular shape of which a front end is disposed near the center display unit 7A and the other display units 7C to 7M are formed in circular shapes, so that the display unit 7B can be easily distinguished from the other display units 7C to 7M.

Further, in the embodiment, although the other display units 7C to 7M are formed in circular shapes, the shapes of the other display units 7C to 7M are not limited to the circular shapes and may be formed in, for example, other shapes such as a rectangular shape to be easily distinguished from the display unit 7B.

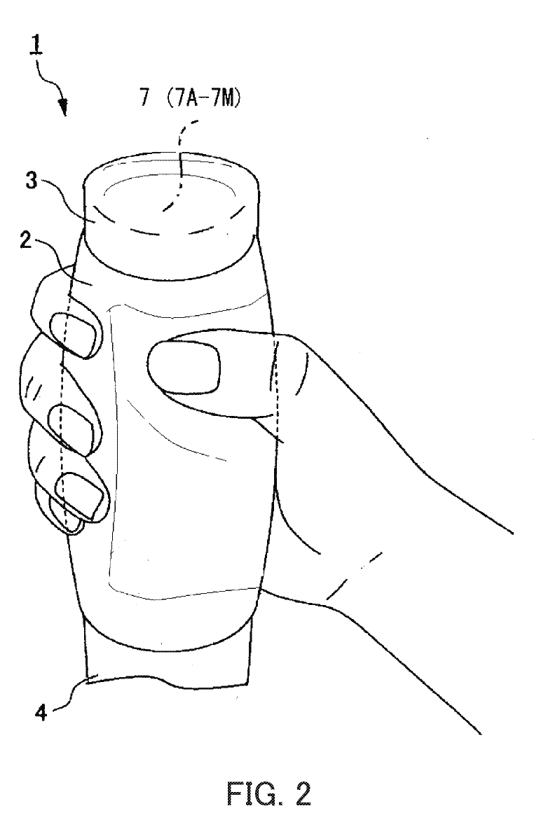

In the grip strength detection mechanism 1 with the above-described configuration, as illustrated in FIG. 2, it is possible to detect and measure a grip strength by gripping and pressing the gripping part 2 while the display unit 7 is directed upward so that the display unit 7 can be visually checked and to perform training by using a detection result of the grip strength.

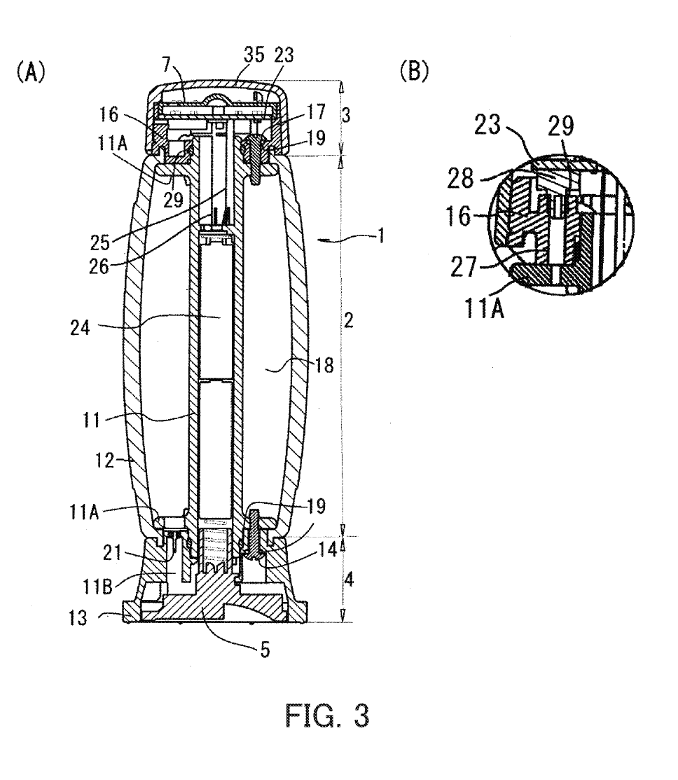

FIG. 3 is a cross-sectional view taken along a line A-A′ of FIG. 1. The grip strength detection mechanism 1 includes a cylindrical battery casing 11 of which both ends are provided with flanges 11A and 11A. Further, the grip part 12 is formed in a cylindrical shape by a flexible material such as silicon resin so that a diameter of the center portion becomes the largest, the battery casing 11 is disposed inside the grip part 12 in the axial direction, an end portion on the side of the distal end portion 4 in the grip part 12 is pressed and sealed to the flange 11A of the battery casing 11 by an end cap 13 corresponding to a cylindrical sealing member located at the distal end portion 4 using a screw 14, and an end portion on the side of the distal end portion 3 in the grip part 12 is pressed and sealed to the flange 11A of the battery casing 11 by a top cap 16 corresponding to a cylindrical sealing member located at the distal end portion 3 using a screw 17. Accordingly, the inside of the grip strength detection mechanism 1 is formed as a cylindrical deformable sealed space 18 by a battery casing 11 and a grip part 12.

Additionally, in the grip strength detection mechanism 1, an O-ring 19 corresponding to a flexible sealing material formed of synthetic rubber, for example, silicon resin is appropriately disposed between the flange 11A of the battery casing 11 and the top cap 16, between the flange 11A of the battery casing 11 and the end cap 13, between the screw 14 and the end cap 13, and between the screw 17 and the top cap 16, so that the leakage of air from the sealed space 18 is prevented. Further, a through-hole 11B is provided in the flange 11A of the battery casing 11 on the side of the end cap 13 and a check valve 21, for example, an umbrella valve 21 is provided on the side of the sealed space 18 in the through-hole 11B, so that the leakage of air can be prevented for a long period of time while preventing an abrupt inflow/outflow of atmosphere with respect to the sealed space 18 [FIG. 3(A)]. Additionally, the O-ring 19 and the umbrella valve 21 may be omitted when the sealed space 18 can be sufficiently maintained for practical use.

Further, the wiring board 23 is disposed at the end portion on the side of the top cap 16. Further, battery terminals 25 and 26 are provided so that the battery 24 can be accommodated inside the cylindrical battery casing 11 after the switch part 5 is separated, an electrode on the side of the switch part 5 in the battery 24 accommodated in this way can be connected to the wiring board 23 via the switch part 5, and an electrode of the battery 24 on the side of the wiring board 23 can be connected to the wiring board 23. Accordingly, in the grip strength detection mechanism 1, the battery 24 is accommodated after the switch part 5 is separated and an electronic circuit provided on the wiring board 23 is driven by the battery 24.

As illustrated in FIG. 3(B), a through-hole 27 is provided in the flange 11A on the side of the wiring board 23 in the battery casing 11 and a pressure sensor 28 is provided on the wiring board 23 so as to block the through-hole 27. Here, the pressure sensor 28 is formed so that an end portion on the side of the flange 11A extends from a cylindrical outer periphery, the cylindrical portion is disposed to be fitted to the through-hole 27, and an O-ring 29 corresponding to a sealing material is disposed between the cylindrical portion and the through-hole 27. Accordingly, the grip strength detection mechanism 1 detects a pressure of the sealed space 18 by the pressure sensor 28 by effectively preventing the leakage of air. Here, the pressure sensor 28 is a so-called semiconductor pressure sensor 28, and various pressure sensors can be widely applied if necessary.

As the pressure sensor 28 used in the invention, for example, a piezoresistive type semiconductor pressure sensor using a piezoresistive effect is suitable.

A sensor chip used in the piezoresistive type semiconductor pressure sensor has a structure in which a silicon layer having a substantially downward concave shape in a cross-section and provided with a small hollow portion formed by etching (this hollow portion serves as a diaphragm (pressure receiving portion)) is laminated on a glass base and a piezoresistive portion (strain gauge) is formed on a thin silicon surface corresponding to the roof portion of the hollow portion.

In the invention, according to the piezoresistive type semiconductor pressure sensor, when the thin silicon surface on the hollow portion (diaphragm) is deflected by the pressure in the sealed space 18 increased as the volume in the sealed space 18 decreases, the grip part 12 gripped by the user is depressed, the electric resistance value of the piezoresistive portion (strain gauge) changes, and this change is converted into an electric signal (this change is processed and amplified by an electronic circuit).

Further, in the grip strength detection mechanism 1, a drive circuit driving the pressure sensor 28, a control circuit processing a detection result of the pressure sensor 28 by a computer, and the light emitting diodes 8A to 8M corresponding to the display units 7A to 7M are provided on the wiring board 23.

In the grip strength detection mechanism 1, the distal end portion 3 is formed in such a manner that a bottomed cylindrical outer cap 35 having a high circumferential wall formed by transparent or translucent resin is fitted to the outside of the top cap 16 to cover the display unit 7 so that the light emitted from the light emitting diodes 8A to 8M through the display unit 7 attached to cover the wiring board 23 can be viewed from the outside.

Both ends of the grip part 12 formed by the cylindrical flexible member in this way are sealed by the end cap 13 and the top cap 16 corresponding the sealing member to form the deformable sealed space 18 surrounded by the grip part 12 and a change in pressure of the sealed space 18 is detected by the pressure sensor 28 held by the sealing member and is displayed.

Further, since the end portions of the grip part 12 are pressed and sealed to the flanges 11A of the battery casing 11 by the cylindrical battery casing 11 having the flanges 11A and 11A formed at both ends and the battery 24 is accommodated inside the battery casing 11, the grip strength detection mechanism can be portably carried and can be further decreased in size and weight. Further, since the grip strength detection mechanism is formed in a cylindrical shape on the whole, an excellent design can be obtained.

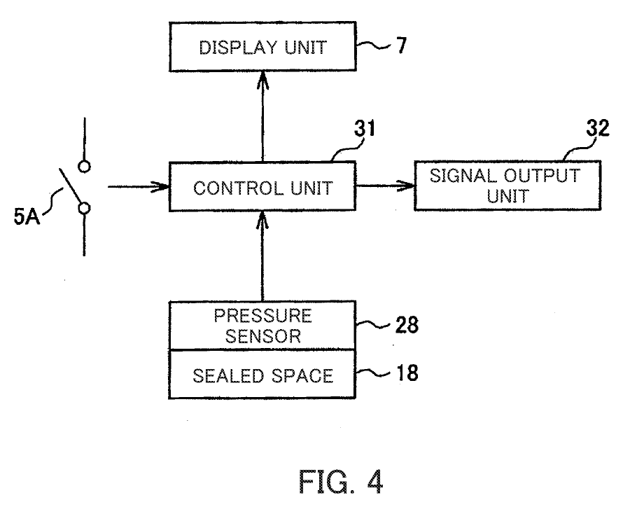

FIG. 4 is a block diagram illustrating a configuration of the grip strength detection mechanism 1. In the grip strength detection mechanism 1, the supply of power starts by the operation of the power switch using the switch part 5 so that components start to be operated, an overall operation is controlled by a control unit 31 corresponding to a computer provided on the wiring board 23 so that a grip strength is detected and measured and a display of the display unit 7 is changed to allow the user to perform training based on the detection/measurement result, and a signal output unit 32 is driven to generate a signal sound.

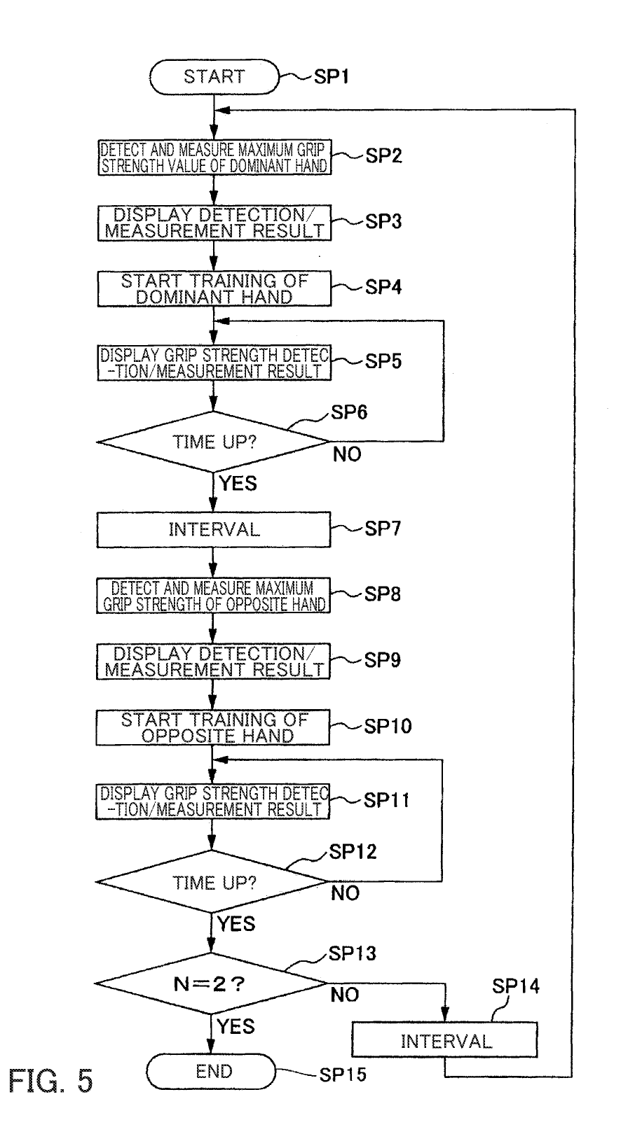

FIG. 5 is a flowchart illustrating a process relating to an operation of the exercise apparatus and a control of the control unit 31. When the control unit 31 starts to be operated by the supply of power, in step SP2, first, a maximum value of the grip strength of a dominant hand (in the embodiment, a right hand is set as a dominant hand and the same applies to the following description) is detected and measured. Here, in the detection/measurement process, first, a signal sound of notifying the start of the detection/measurement is generated by the driving of the signal output unit 32 and then a pressure of the sealed space 18 is repeatedly detected and measured by the pressure sensor 28. When an increase in pressure of the sealed space 18 is detected based on the detection/measurement result, the control unit 31 determines that the gripping part 2 is strongly gripped by the user and the grip strength of the user starts to be measured. Further, after this determination, a maximum pressure immediately before releasing the gripping part after gripping the gripping part by the user with a full strength is detected and this detected pressure is recorded as the maximum grip strength of the dominant hand (right hand) relating to training.

Hereinafter, an embodiment of the invention will be described.

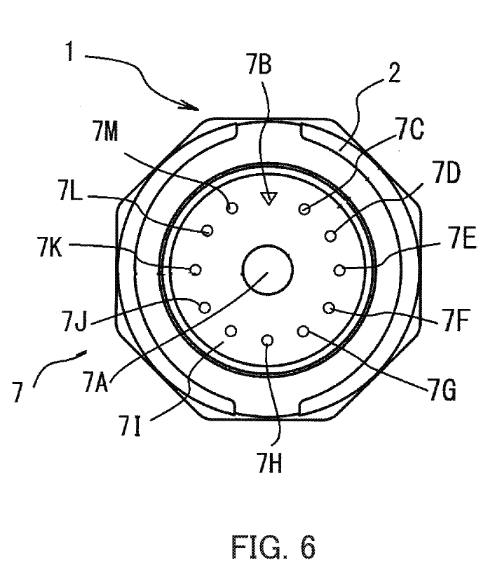

When the grip strength is detected and measured in this way, the control unit 31 may display the detection/measurement result on the display unit 7 (step SP3). Here, as illustrated in FIG. 6, the control unit 31 increases the number of the display units emitting light, for example, whenever a measurement value increases by a certain value in the clockwise direction from the display unit 7B based on the display unit 7B having a different shape among the display units 7B to 7M arranged at the same angle intervals. In the embodiment, when the certain value is set to 5 kg and the grip strength corresponding to the detected pressure is equal to or larger than 0 kg and smaller than 5 kg, only the display unit 7B having a different shape is turned on. Further, when the detected and measured grip strength is equal to or larger than 5 kg and smaller than 10 kg, the display unit 7C which is next to the display unit 7B having a different shape in the clockwise direction is turned on. Furthermore, when the detected and measured grip strength is equal to or larger than 10 kg and smaller than 15 kg, two display units 7C and 7D which are next to the display unit 7B having a different shape in the clockwise direction are turned on. Additionally, step SP3 can be appropriately omitted.

Additionally, the grip strength detection mechanism 1 displays the grip strength measurement/detection result in a gradual display. Furthermore, in the display of the grip strength measurement/detection result (step SP3), a display unit capable of displaying numbers may be provided to display information by numbers and also sound may be used to notify information. When the grip strength is displayed in this way, the control unit 31 drives the signal output unit 32 to generate a signal sound and to call the attention of the user.

Next, the control unit 31 waits for the elapse of a certain interval time (for example, 1 minute) while displaying the detection/measurement result of the grip strength in this way and then starts the training of the hand (in this case, the right hand) provided for detecting and measuring the grip strength (step SP4). Here, the control unit 31 notifies the start of training to the user by driving the signal output unit 32 to generate a signal sound. Additionally, the interval time can be appropriately omitted and the training may be immediately started without counting the interval time of 1 minute. Then, the grip strength is repeatedly detected and measured by the pressure sensor 28. The control unit 31 sets a certain percentage of a strength of the grip strength detected and measured in step SP2 (that is, a pressure equal to or smaller than the maximum grip strength detected and measured in step SP2) to a reference grip strength and determines the detection/measurement result repeatedly acquired by the reference grip strength. Then, the control unit 31 determines whether the detected and measured grip strength exists within a certain range of the reference grip strength and displays the determination result (step SP5).

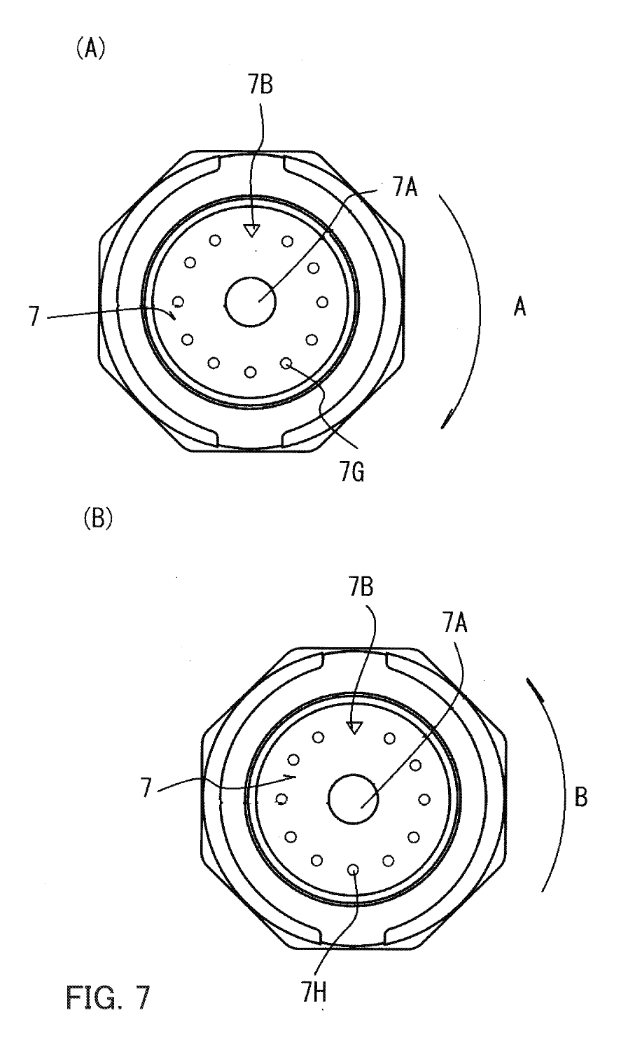

Here, the reference grip strength is set to, for example, about 30% of the grip strength detected and measured in step SP2. The control unit 31 sets, for example, a pressure of ±5 kg within a range of a certain height difference degree with respect to the reference grip strength to a pressure of an upper limit value and a lower limit value and performs a determination one by one by the upper limit value and the lower limit value. Here, as illustrated in FIG. 7(A), when the detection/measurement result is equal to or larger than the upper limit value, the control unit 31 displays the display unit 7A in red at the center display unit 7A of the display unit 7. On the contrary, when the detection/measurement result is equal to or smaller than the lower limit value, the control unit displays the center display unit 7A in yellow. Further, when the detection/measurement result is between the upper limit value and the lower limit value, the control unit displays the center display unit 7A in blue. Additionally, the color of the light emitted from the display unit 7A is not limited to the above-described example. For example, blue may be displayed when the measurement result is equal to or larger than the upper limit value, green may be displayed when the measurement result is between the upper limit value and the lower limit value, red may be displayed when the measurement result is equal to or smaller than the lower limit value, and this color may be appropriately changed.

Accordingly, the control unit 31 notifies the magnitude of the current grip strength with respect to the reference grip strength to the user. When a certain time elapses after the training is performed for a certain time (for example, 2 minutes), the control unit 31 drives the signal output unit 32 to generate a signal sound so that the instruction of the release of the gripping of the grip part is notified (step SP6). Additionally, after the training time (2 minutes) elapses, the display units 7B to 7M disposed around the display unit 7A at the same intervals are selectively displayed to notify the current state to the user. More specifically, for example, as indicated by the arrow A of FIG. 7(A), the display units 7B to 7M are selectively turned on or off, a portion provided for the blinking is sequentially changed in the clockwise direction, and the selective blinking is made to rotate one round by the training time so that the user is notified of the current state.

Additionally, regarding the display of the pressure detection result based on the reference grip strength, instead of a display of such three stages of color coding by red, blue, and yellow, a color-coded display may be performed by a color different from these colors or a display of four or more stages of color coding may be performed. Further, a selective display may be performed by the circumferentially arranged display units 7B to 7M or the grip strength display unit may be separately provided and the detected and measured grip strength may be displayed together.

In this way, when the display of the display unit 7A is changed by the reference grip strength, the grip strength detection mechanism 1 can perform the training so that the grip strength is maintained at the reference grip strength for a certain time and the muscles of the fingers are relaxed. Accordingly, it is reported that the high blood pressure can be relieved as described below by such training. That is, when the grip strength is maintained at the reference grip strength for a certain time, the muscles provided for the gripping are safely strained while efficiently preventing the occurrence and deterioration of diseases relating to the high blood pressure and the muscles are relaxed at a stretch when the gripping is released, so that the blood vessel expands. Since the elasticity of the blood vessel is remarkably improved by the repeated training, the high blood pressure is solved. Accordingly, in the embodiment, since the high blood pressure can be prevented and solved by the training using the grip strength detection mechanism, the grip strength detection mechanism can be used for the training aiming at the above-described effect.

In this way, when the gripping is released, the control unit 31 generates a signal of allowing the user to release the gripping and to have a rest time by waiting for a certain interval time (for example, 1 minute) (step SP7). For the interval time of 1 minute, for example, when 1 minute elapses after the display units 7B to 7M which are turned on together are sequentially turned off in the counter-clockwise direction every certain time, the display units 7B to 7M may be turned off together to notify the elapse of 1 minute to the user. Further, when a certain time elapses, the detection and the measurement of the grip strength for gripping the grip part by a hand opposite to a dominant hand (in the embodiment, a hand opposite to a dominant hand is set as a left hand and the same applies to the following description) starts (step SP8). Then, similarly to the description above in step SP2, the grip strength is detected and the grip strength corresponding to the detection pressure is displayed (step SP9).

Additionally, step SP8 and step SP9 can be appropriately omitted.

Further, the training starts by a hand opposite to a dominant hand (step SP10). Similarly to a case of a right hand, the reference grip strength is calculated from the detected and measured grip strength, the detected and measured pressure is determined by a reference pressure, and the detected and measured pressure is displayed (steps SP11 and SP12). Additionally, in this case, the user may recognize the training of the left hand by appropriately displaying the elapse of time while changing the display of the display units 7C to 7M in the counter-clockwise direction as illustrated in FIG. 7(B) by the comparison with FIG. 7(A).

In this way, when the training ends with the elapse of a certain time, the number of times of repeating the training is determined. Then, when the number of times of repeating is smaller than two times, a series of processes are repeated after returning to the detection and measurement of the grip strength of the right hand across the interval (steps SP13 to SP14). When the training has been repeated twice, the overall process ends (step SP15).

Additionally, in the operation of the exercise apparatus of the invention, step SP3, step SP8, and step SP9 can be appropriately omitted as described above.

Further, the reference grip strength is set as a numerical value calculated from the maximum grip strength value of a dominant hand first detected and measured in step SP2 and can be uniformly set. Accordingly, it is possible to omit a trouble of detecting and measuring the maximum grip strength value again. That is, in this case, step SP8 and step SP9 can be omitted along with step SP2, step SP8, and step SP9 of the second training after the training is performed by a dominant hand and the opposite hand (step SP1 to SP14).

Next, an effect of the training using the exercise apparatus with the grip strength detection mechanism 1 of the invention will be described.

The training content is as described in the first embodiment. When only the operation of the exercise apparatus of the invention is repeatedly and simply described, Operations 1 to 4 below are performed after the switch of the main body is turned on.

Operation 1: a hand is released from the grip part 12 in a loosened state after gripping the grip part with a full strength. At this time, a pressure of the sealed space 18 with 100% of the grip strength of the user is detected.

Operation 2: the grip part 12 is gripped again. However, at this time, the grip part is gripped with a strength of about 30% of the maximum grip strength of the user and is gripped with a constant grip strength while the center display unit 7A continuously emits blue light for 2 minutes. Additionally, a case in which the display unit 7A emits blue light while the grip part 12 is gripped displays a case in which the grip strength of the user who grips the grip part is about 30%, a case in which the display unit emits yellow light displays a case in which the grip strength is weak (the grip strength does not reach about 30%), and a case in which the display unit emits red light displays a case in which the grip strength is strong (the grip strength exceeds about 30%).

Operation 3: the grip part is gripped with a strength of about 30% of the maximum grip strength for 2 minutes and is released for 1 minute. Additionally, it is important to surely take a rest after releasing the gripping for 1 minute.

Operation 4: exercises (1 set) that repeat operations 1 to 4 once and repeat these operations four times in total twice with left and right hands.

Additionally, it is effective to use the exercise apparatus at a position lower than a heart at the time of using the exercise apparatus of the invention. Then, it is more effective when gripping the grip part 12 by placing the exercise apparatus of the invention on the thigh while sitting on the chair.

In order to check the effect obtained by the above-described training, ten subjects actually repeated the above-described series of exercise (Operations 1 to 4) using the exercise apparatus of the invention four times (1 set) in total twice with the left and right hands at a pace of 5 days a week for four weeks and the numerical value of the systolic blood pressure of each subject was measured under the supervision of the Exercise Physiology Laboratory at Nippon Sport Science University, Department of Health Science.

Table 1 lists the sex and the age of each subject and the numerical value of the measurement result of the systolic blood pressure of each subject at the first and fourth weeks.

In the examination, the exercise is performed using the exercise apparatus of the invention once a day (8 minutes+3 minutes) as 1 set at a pace of 5 days a week for four weeks as described above without changing the lifestyle so far. Then, the measurement is performed in the Exercise Physiology Laboratory at Nippon Sport Science University in the first week and the fourth week. As described above, 1 set of once (8 minutes+3 minutes) a day means that exercise of “gripping the grip part for 2 minutes and taking a rest for 1 minute” is repeated four times in total twice with the left and right hands while gripping the exercise apparatus of the invention with 30% of the grip strength in the left and right palms.

TABLE 1

| | | Those | | | | |
|---|---|---|---|---|---|---|
| | | who used | | | | |
| | | anti- | | | | |
| | | hypertensive | | | | |
| | | agent | First | Fourth | (Reduction | |
| Age | Sex | ⋆ | week | week | value) | (Unit MhG) |
| 69 | Female | | 160 | 131 | (29) | |
| 46 | Male | | 168 | 145 | (23) | |
| 57 | Male | | 180 | 168 | (12) | |
| 44 | male | | 154 | 144 | (10) | |
| 54 | Male | | 167 | 159 | (8) | |
| 76 | Male | ⋆ | 122 | 114 | (8) | |
| 68 | Male | ⋆ | 146 | 140 | (6) | |
| 64 | Female | | 141 | 136 | (5) | |
| 48 | Female | | 137 | 133 | (4) | |
| 60 | male | ⋆ | 123 | 121 | (2) | |
| | | | | | (10.7) | Average |
| | | | | | | decrement value |

As obvious from Table 1, it was checked that the systolic blood pressure of all ten subjects decreased in four weeks. Further, it was also checked that there was a decrease in systolic blood pressure also for those who used antihypertensive agents.

The reason why the systolic blood pressure has decreased is still under study. Although the blood circulation (the flow of blood) is slightly lowered since the muscles of the arm compress the blood vessel when the exercise apparatus of the invention is gripped with about 30% of the grip strength for 2 minutes, the muscles are simply loosened. When taking a rest for next 1 minute, accordingly, the blood circulation inside the blood vessel is also activated, the cell inside the blood vessel is stimulated, and NO (nitric oxide) is actively secreted from the inner wall of the blood vessel by the stimulus. Accordingly, the above-described reasons can be rationally predicted.

That is, NO (nitric oxide) is made of thin vascular endothelial cells on the inner wall of the blood vessel, spreads in the blood vessel, improves the blood circulation, keeps the blood vessel soft, and has a function of stabilizing the blood pressure.

Further, when the function of the thin vascular endothelial cells decreases due to aging, disorder of lifestyle habits, stress, or the like, the secretion of NO (nitric oxide) decreases. As a result, the blood pressure tends to rise. Here, according to the obtained knowledge, when the exercise apparatus of the invention is gripped with about 30% of the grip strength with both hands while alternately taking a rest, NO is secreted from the blood vessel of both arms and goes to the whole blood vessel, so that the blood pressure is lowered.

Further, as shown in Table 1, there was also an effect for those who were taking antihypertensive agents. It seems that NO has a function of softening the blood vessel and decomposing the plaque which may clog the blood vessel.

Although a detailed configuration suitable for the embodiment of the invention has been described above, the configuration of the embodiment of the invention can be modified into various forms in the scope not departing from the spirit of the invention and can be combined with the conventional configuration.

That is, in the above-described embodiment, although a case has been described in which about 30% of the detected and measured maximum grip strength is set to the reference grip strength, the invention is not limited thereto. For example, the reference grip strength can be modified variously within the range in which the gripping continued for a certain time does not give a burden on the human body. Further, the upper limit value and the lower limit value relating to the determination can be also set variously in the same way.

Further, in the above-described embodiment, although a case has been described in which the grip strength is detected and measured and the reference grip strength is set and recorded for each training, the invention is not limited thereto. For example, when the reference grip strength can be sufficiently applied to the practical use, the setting of the reference grip strength may be omitted for each training.

Further, in the above-described embodiment, although a case has been described in which the training relating to the grip strength of the right hand and the training relating to the grip strength of the left hand are performed as a continuous process by the control unit, the invention is not limited thereto. For example, both trainings may be performed individually.

Further, in the above-described embodiment, although a case has been described in which the grip strength is detected and measured and the training relating to a high blood pressure prevention is performed, the invention is not limited thereto. For example, the detection/measurement of the grip strength and the training may be respectively performed by changing a mode and the invention can be also applied to a case in which only the grip strength is simply detected and measured.

### EXPLANATION OF REFERENCE NUMERALS

1 GRIP STRENGTH DETECTION MECHANISM
2 GRIPPING PART
3, 4 DISTAL END PORTION
5 SWITCH PART
7, 7A to 7M DISPLAY UNIT
8A to 8M LUMINOUS BODY (LED)
10 EXERCISE APPARATUS
11 BATTERY CASING
11A FLANGE
11B THROUGH-HOLE
12 GRIP PART
13 END CAP (SEALING MEMBER)
14, 17 SCREW
16 TOP CAP (SEALING MEMBER)
18 SEALED SPACE
19, 29 O-RING
21 CHECK VALVE (UMBRELLA VALVE)
23 WIRING BOARD
24 BATTERY
25, 26 BATTERY TERMINAL
27 THROUGH-HOLE
28 PRESSURE SENSOR
30 COVER
31 CONTROL UNIT
32 SIGNAL OUTPUT UNIT
35 OUTER CAP

1. A grip strength detection mechanism comprising:
a grip part which is formed by a cylindrical flexible member;
a sealing member that seals both ends of the grip part and forms a deformable sealed space surrounded by the grip part;
a pressure sensor which is provided inside the sealed space and detects a change in pressure inside the sealed space deformed by a grip strength of a user who grips the grip part; and
a control unit which displays a predetermined pressure detected by the pressure sensor on a display unit.

2. The grip strength detection mechanism according to claim 1,
wherein a battery casing having a flange formed at both ends is provided inside the grip part, and
wherein both ends of the grip part are respectively pressed against both flanges by the sealing member so that the grip part is sealed by the sealing member.

3. The grip strength detection mechanism according to claim 1,
wherein the control unit sets a predetermined pressure equal to or smaller than a pressure detected by the pressure sensor as a reference grip strength based on the detected pressure by a maximum grip strength of a user who grips the grip part, and
displays a detection result in which the grip strength of the user who grips the grip part reaches the reference grip strength on the display unit.

4. The grip strength detection mechanism according to claim 3,
wherein the reference grip strength is set within a range of a certain height difference degree with respect to a pressure corresponding to the reference grip strength and the reference grip strength is displayed on the display unit.

5. An exercise apparatus comprising:
the grip strength detection mechanism according to claim 1.

6. A method for using the exercise apparatus including the grip strength detection mechanism according to claim 1, comprising:
repeating an operation of gripping the grip part for a certain time with a reference grip strength and releasing the gripping for a certain time.

7. The method for using the exercise apparatus according to claim 6, further comprising:
setting the reference grip strength to about 30% of the maximum grip strength of the user; and
repeating an operation of gripping the grip part with a strength of about 30% of the maximum grip strength of the user for 2 minutes and releasing the gripping for 1 minute so that the operation is performed twice by each of left and right hands and is performed four times in total.
